(19) United States
(12) Patent Application Publication
(10) Pub. No.: US 2013/0218023 A1
(43) Pub. Date: Aug. 22, 2013

(54) METHODS FOR DIAGNOSIS OF COLORECTAL CANCER

(71) Applicants: Francis M. Giardiello, Sparks, MD (US); Daniel L. Edelstein, Phoenix, MD (US); Jessica Ramella-Roman, Washington, DC (US); Linda M. Hylind, Fallston, MD (US); Ali Basiri, Silver Spring, MD (US)

(72) Inventors: Francis M. Giardiello, Sparks, MD (US); Daniel L. Edelstein, Phoenix, MD (US); Jessica Ramella-Roman, Washington, DC (US); Linda M. Hylind, Fallston, MD (US); Ali Basiri, Silver Spring, MD (US)

(73) Assignee: THE JOHNS HOPKINS UNIVERSITY, Baltimore, MD (US)

(21) Appl. No.: 13/639,891

(22) PCT Filed: Apr. 8, 2011
(86) PCT No.: PCT/US11/31854

§ 371 Date: May 2, 2013

Related U.S. Application Data
(60) Provisional application No. 61/322,075, filed on Apr. 8, 2010.

(51) Int. Cl. A61B 5/00

(52) U.S. Cl. CPC A61B 5/7282; A61B 5/0088; A61B 5/4255; A61B 5/7275; A61B 5/489; A61B 5/4848
USPC 600/476

(57) ABSTRACT

Methods and systems for use of phenotypic markers, principally oral mucosal vascular density alone or in combination with detection of other markers, to identify individuals afflicted with or having an increased risk of hereditary colorectal cancer, especially familial adenomatous polyposis, are disclosed.

### BACKGROUND OF THE INVENTION

1. Field of the Invention

The present invention relates generally to methods and systems for detecting the presence of or risk of developing cancer and more specifically to methods for detecting hereditary colorectal cancer (HCC), especially familial adenomatous polyposis (FAP).

2. Background Information

Colorectal cancer, also referred to as colon cancer or rectal cancer, is a major American health problem. Colorectal cancer includes any cancer in the colon from the beginning (at the cecum) to the end (at the rectum). Colorectal cancer occurs when cells that line the colon (large bowel, large intestine) or the rectum (lower portion of the colon) become abnormal and grow out of control. Polyps, which are usually benign growths that protrude from a mucous membrane, can form in the colon and rectum. Such adenomatous polyps can eventually progress into cancer if left untreated.

Most colorectal cancers are sporadic, that is individuals developing such colorectal cancers have no prior family history of the disease. A number of different inherited conditions, however, can give rise to a significant risk of colon cancer. Individuals with a family history of colorectal cancer are described as having familial or hereditary colorectal cancer. It is estimated that 15% to 30% of colorectal cancers are familial. A single gene, a combination of genes, or a combination of genetic and environmental factors can cause familial colorectal cancer. Typically, such families have one or two members with a history of colorectal cancer or precancerous polyps.

A family is considered to have hereditary colorectal cancer when the exact gene that causes the disease is known. Hereditary colorectal cancer (HCC) is thought to be among the most common of inherited human disorders with an estimated frequency of 1/500 individuals. About 3 to 5% of those individuals afflicted with colorectal cancer have FAP or HNPCC. Others may have familial colorectal cancer (FCC).

Familial adenomatous polyposis (FAP) is a disorder that leads to hundreds, even thousands, of polyps in the colon and rectum at a young age, usually as a teenager or young adult. FAP also is referred to as hereditary polyposis of the colorectum, familial polyposis, and Gardner's syndrome. It can progress to colorectal cancer in young adulthood if colectomy is not performed. This disorder is characterized by the development of hundreds of colorectal adenomas in adolescence. Bussey H. J. R., Familial polyposis coli. Family studies, histopathology, differential diagnosis, and results of treatment. Johns Hopkins University Press: Baltimore, Md. (1975). Nearly all affected individuals will develop colorectal cancer by the 6th decade of life if prophylactic colectomy is not performed. Id. Presymptomatic genetic testing of at-risk individuals can differentiate affected members from those unaffected by FAP. Giardiello et al., Familial adenomatous polyposis. Eds. Genetic testing, however, is costly and deleterious mutations are not found in all families with this clinical diagnosis.

FAR is one of two well-described forms of HCC. The other is hereditary nonpolyposis colorectal cancer (HNPCC), also referred to as Lynch syndrome or cancer family syndrome, is a condition in which the tendency to develop colorectal cancer is inherited. A mutation in the genes hMLH1 and hMSH2, hMSH6, and hPMS2, which when functioning normally would protect against colon cancer, is the known cause of HNPCC. Individuals afflicted with HNPCC have a 50% chance of passing the HNPCC gene to each of their children.

Presently, patients afflicted with HCC or FCC are typically diagnosed by reviewing family history, i.e., identifying multiple members afflicted with colorectal cancer, and/or by molecular testing of the tissue or blood of patients suspected by clinical or family history characteristics to have this condition. The usual diagnosis of FCC/HCC typically involves expensive molecular testing, which can cost from $750 to $3,000 with a mutation pickup rate of about 60%. Such testing also can be lengthy. For example, it can take from about 6 weeks to about 10 weeks to obtain a result. This time-consuming process often interferes with proper treatment of patients afflicted with colorectal cancer who need medical decision making to be done expeditiously.

Thus, there remains a need for a diagnostic method for detecting and/or screening for the presence of HCC, especially FAP, and/or the risk of developing FAP. There further remains a need for a method to readily differentiate FAP in a patient from other forms of HCC (e.g., HPNCC) and from FCC.

### SUMMARY OF THE INVENTION

In one aspect, the presently disclosed subject matter is directed to methods and devices for using of phenotypic markers, especially oral mucosal vascular density (OMVD), for diagnosing colorectal cancer or an increased risk of colorectal cancer in a subject, especially FAP.

In another aspect, the presently disclosed subject matter is directed to methods and devices for differentiating between FAP and HNPCC in subjects having or at risk of having HCC.

In another aspect, the presently disclosed subject matter is directed to methods and devices for differentiating between FAP and FCC in subjects having or at risk of having colorectal cancer.

In other embodiments, the oral mucosal reflectance of a subject identified as a probable risk for FAP can be screened as a confirmation that it, rather than HPNCC, is likely present.

In another aspect, the invention provides an apparatus for measuring the oral vascular density in a subject.

In a further aspect, the invention provides a method to monitor changes in vascular density over time; e.g., responses over the course of cancer treatment, such as anti-angiogenic therapy.

In another aspect, the present invention provides a kit for measuring the oral vascular density in a subject. The kit includes reagents for performing the measurements. The kit can also include instructions on using kit components to identify an increased risk of developing FAP.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Figures, which are not necessarily drawn to scale.

FIG. 1 is a schematic of an image capture and analysis system useful in the invention.

FIG. 2 is a scatter plot showing the values for oral mucosal vascular density measured in individuals afflicted with familial adenomatous polyposis (FAP) and controls.

FIG. 3 is a scatter plot showing the values for oral mucosa reflectance measured in individuals afflicted with familial adenomatous polyposis (FAP) and controls.

FIG. 4 shows receiver operator characteristic (ROC) curves for oral mucosal vascular density for familial adenomatous polyposis (FAP) patients over a range of cutoff points, indicative of the sensitivity and specificity provided, by the FAP test of the invention.

FIG. 5 shows the age relationship between measured OMVD values (scale of 10-6) and patient age in control subjects (o), FAP positive (¥) and FAP negative (+) patients.

FIG. 6 shows the ratio (R2 at 650 nm wavelength/R1 at 550 nm) of OMR values in control subjects (o), FAP positive (¥) and FAP negative (+) patients.

### DETAILED DESCRIPTION OF THE INVENTION

General Caveats

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Methods, Devices and Kits for Identifying FAP with Oral Vascular Density as a Marker

Toward expediting diagnosis of HCC, prior researchers have focused on whether the presence of HNPCC in a patient can be determined by changes in oral mucosal reflectance (OMR). DeFelice, et al., have suggested that OMR is a sufficient phenomenon to be useful as a clinical marker for HNPCC (Gut, 52:1764-1767, 2003). Given the hereditary similarities between FAP and HNPCC, one might reasonably presume that OMR would be diagnostic for FAP as well. Surprisingly, the inventors have determined that it is not. Instead, while changes in OMR don't occur at diagnostically significant levels in FAP patients, changes in oral mucosal vascular density (OMVD) do. The invention therefore provides a method for assaying changes in vascular density to diagnose the presence or risk of developing FAP. The methods may be performed non-invasively, quickly and are susceptible to automation, as hereinbelow described.

The invention may also be used in conjunction with other diagnostic modalities for colorectal cancer; e.g., colonoscopy. As to FAP in particular, several other phenotypic markers have been identified, albeit ones identifiable through invasive assay techniques, such as biopsy and subsequent laboratory examination, with lower efficacy rates than may be obtained through use of the invention. Such phenotypic markers include occult radio-opaque jaw lesions, which are small, usually multiple, well circumscribed radiodensities detected by panoramic x-rays in the premolar and molar regions of the mandible and maxilla. See Utsunomyia J., and Nakamura T., “Osteomatous changes and tooth abnormalities found in the jaws of patients with familial polyposis coli,” Br. J. Surg. 62:45-51 (1975).

A second phenotypic marker is congenital hypertrophy of the retinal pigment epithelium (CHRPE). These are discrete, round to oval, darkly pigmented retinal lesions ranging from 0.1 to 1.0 optic disc diameters in size detected by indirect ophthalmoscopy. See Bosman et al., WHO Classification of Tumours of the Digestive System, 4th edition (2009); Traboulsi et al., “Pigmented ocular fundus lesions: Prevalence and significance in Gardner syndrome,” N. Engl. J. Med. 316:661 667 (1987). The efficacy of either CHRPE or occult radio-opaque jaw lesions for identifying patients afflicted with FAP is 70% and 67%, respectively, and 80% when both markers are used in combination. See Giardiello et al., “The value of combined phenotypic markers in identifying inheritance of familial adenomatous polyposis,” Gut 32:1170-4 (1991). The invention provides a test for FAP with efficacy greater than 80% and up to 90%.

Further, the OMR of a subject identified as a probable risk for FAP can be screened as a confirmation that it, rather than HPNCC, is likely present.

A method according to the present invention can be performed during routine clinical care, for example as part of a general regular checkup, on a subject having no apparent or suspected neoplasm such as cancer. Therefore, the present invention in certain embodiments, provides a screening method for the general population. The methods of the present invention can be performed at a younger age than present cancer screening assays, for example where the method can be performed on a subject under 65, 55, 50, 40, 35, 30, 25, or 20 years of age.

In the present invention, the subject is typically a human but also can be any mammalian organism, including, but not limited to, a dog, cat, rabbit, cow, bird, rat, horse, pig, or monkey.

As mentioned above, for certain embodiments of the present invention, the method is performed as part of a regular checkup. Therefore, for these methods the subject has not been diagnosed with cancer, and typically for these present embodiments it is not known that a subject has a colorectal cancer.

Once the OMVD has been measured for a particular patient, the risk of having cancer is then assessed by comparing the measured values to a known relationship between the OMVD present and the probability of the presence of the particular type of cancer, especially FAP. Such relationship may be identified, as illustrated in the Examples, by determining OVMD measurements in a statistically meaningful manner for FAP patients, and determining the OVMD measurements in a statistically meaningful manner in patients without cancer, and then calculating an odds ratio as a function of the measurements at which FAP is statistically likely to be present or develop.

Those of ordinary skill in the art will be familiar with or can readily ascertain means for measuring OMVD. For example, Greene and Rieder describe a method for use of rhodamine-labeled Griffonia simplicifolia (GS-I) lectin to define both perfused and unperfused microvessels ranging from small arteries (20 μm) to normal capillaries (3-6 μm) to venous capillaries (6-9 μm), combined with a computerized image processing technique that rapidly and automatically determines vascular density in tissue sections (Methods Mol Med., 51:489-96 (2001)). Haney, et al. describe a MRI-based method for image capture and analysis of vascular density (Mol. Imaging, 2011 Mar. 28. [Epub ahead of print]). However, the invention may be practiced using a relatively simple image capture technique coupled with a robust numerical analysis computer program evaluation of the resulting data.

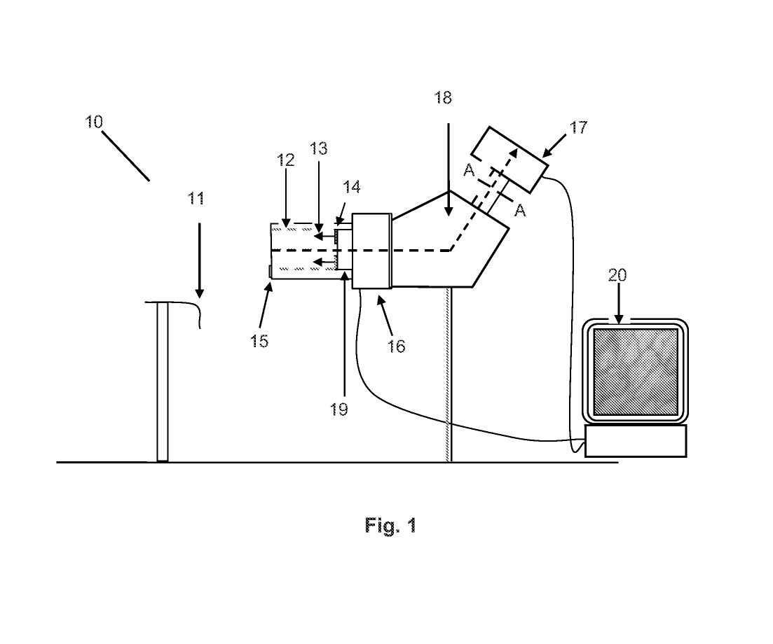

An exemplary imager system 10 for use in performing the OVMD measurements of the invention (which may also be employed for OMR measurement) is schematically shown in FIG. 1. Briefly, the system includes a chin rest 11, a stabilizer tube 12, a light source 13 (preferably mounted with a polarizing filter 14 and magnifying optics (not shown)), a pressure sensor 15, a tunable filter 16, a detector device (e.g., camera) 17 connected to a microscope 18 with illuminator 19 capable of recording an image of light reflected from the subject; and, a numerical analysis calculation program provided on a computer system 20 for calculating of a mucosal vascular density from a selected portion of the image by tracing each vessel in the mucosa with an automatic tracing algorithm.

The liquid crystal tunable filter (LCTF) 16 is commercially available; e.g., from VariSpec LCTF, Cambridge Research and Instrumentation, Inc., Woburn, Mass. The LCTF 16 has an operational range of 400 to 720 nm in the visible spectrum with a 7 nm bandwidth and utilizes electronically controlled liquid crystal elements to select a transmitted wavelength range while blocking all others. This fine resolution allows for the precise isolation of individual wavelengths, providing a rapid, vibrationless selection of any wavelength in the visible to Near Infrared (NIR) range (470 to 700 nm). The LCTF 16 may be used to record reflected light at increments of 5 nm within a range of 500 nm to 700 nm.

Camera 17 (e.g., the 8 bit scientific camera available from Lumenera Corp., Ottawa, ON, Canada) mounted to a stereo microscope 18 captures magnifies images at a frame rate of 30 fps with a size of 1392×1040 pixels. Other suitable image capture devices for use in the invention are commercially available; e.g., CCD or CMOS digital cameras used for microscopy (such as the INFINITY® line of cameras available from Lumenera Corp., Ottawa, ON, Canada). Conveniently, a camera compatible for use with the MATLAB® analysis program (e.g., a MATLAB® camera, Mathworks, Natick, Mass.) might be used so images captured can be saved for analysis using the MATLAB® program, described further below.

Camera 17 is connected to microscope 18; e.g., through an eye-piece optical adaptor with a ring illuminator (Newport, Irvine, Calif.) 19 positioned in the acquisition side of the microscope assembly. Polarized filter sheet 14 (e.g., as available through Edmund Optics, Barrington, N.J.) may be positioned in front of light source 13 and aligned perpendicularly to the LCTF 15. Such cross-polarization imaging not only eliminates specular reflection from the first interface but also minimizes the acquisition of single scattering photons remitted by the superficial tissue. Only photons that have undergone multiple scattering events are allowed back through polarizer 14 to camera 17. Some of these photons will travel through the superficial vasculature on their way back to imager system 10 in a process of transillumination. The high absorption of hemoglobin in the vasculature will then increase the contrast of these vessels compared to the avascular background.

The detector and the numerical analysis program (e.g., one loaded onto a microprocessing environment such as a computer 20 shown in FIG. 1) are preferably in electronic communication for automatic transmission of the image data from the detector to the program. In such a system, the image acquisition and data analysis may be conveniently automated.

Images obtained on imager system 10 are transmitted to computer 20 (along line A-A on FIG. 1) and stored for later analysis. Conveniently, the analysis can be preformed through use of commercially available numerical analysis software programs, such as MATLAB® (Mathworks, Natick, Mass.) and other programs from Mathematica, Maple, IDL™ by ITT Visual Information Solutions and Metlynx, as well as open source programs such as GNU Octave™, FreeMat™, and Scilab™.

Used according to the invention, the system provides OVMD values with a sensitivity and specificity (efficacy) at or above 80% and up to 90%. An alternative system wherein the vessel tracing is performed manually may also be employed with efficacy lower than the system having an automatic tracing program, but still at or above 80%.

To perform the method of the invention, images are acquired from the inside of each patient's lip (conveniently, the lower lip). Individual images may be acquired in an exposure time of as little as 250 milliseconds (ms) or longer, with a constant focal length. Multiple images are preferably obtained for one or more sets of images to be used in analysis; e.g., from 1 to 120 images per set, preferably between 100 and 120 images per set, with each such set being obtained within a timeframe of 5 to 60 seconds; for example, between 10 and 50 seconds, 15 and 45 seconds, 20 and 40 seconds, or 25 to 30 seconds. Patients may relax their lips between acquisition of image sets.

Briefly, with reference to the schematic of the imager system 10 of FIG. 1, images are obtained with the patient's chin positioned inside the chin holder 11 of an image capture device 10. To prepare for image acquisition, the patient manually pulls their lower lip downward using both hands over a stabilizer apparatus 12 positioned perpendicular to the chin rest while remaining as still as possible, or the position can be mechanically manipulated (e.g., using forceps). An imaging field of about 1 cm in diameter is sufficient, although larger imaging samples of 1.25, 1.5, 1.75 and 2 cm may also be utilized. Images are obtained in the near infrared spectrum of between 470 and 700 nm, preferably between 500 and 700 nm, or 550 and 700 nm, or 575 nm and 700, or at 550 nm, or at 650 nm. In a particularly preferred approach, multiple images are obtained at multiple wavelengths, starting at an initial wavelength of about 500 nm and increasing in increments of about 5 nm to a final wavelength of about 700 nm.

To calculate OMVD, a portion of each image (e.g., a 300×600 pixel section is sufficient for use with the MATLAB® analysis program) is selected and each vessel in the image traced using a suitable automatic tracing program or by manual measurement. Conveniently, the automatic tracing algorithm disclosed in Sofka M. and Stewart C. V., “Retinal vessel centerline extraction using multiscale matched filters, confidence and edge measures,” IEEE Transactions on Medical Imaging 25:1531-1546 (2006) may be employed. A binary map of the traced vessels is generated for analysis. For example, an algorithm quantifying the Kolmogorov Complexity of traced images may be employed to calculate an oral vascular density score for each subject (see Kaspar F., and Schuster H. G., “Easily calculable measure for the complexity of spatiotemporal patterns,” Physical Review A 36:842 (2005)). Use of an extended template of a multiscale matched filter helps to preserve vessels that are only a pixel wide and usually low contrast with respect to background in images.

According to the invention, increased OMVD values compared to control values (which may be provided as part of a kit, as noted hereinbelow) are considered to be diagnostic for FAP. Patients afflicted with FAP are those with statistically significantly increased oral mucosal vascular density compared to controls. In this respect, the analysis will ideally account for differences in demographic characteristics between subjects with FAP and controls, using a two-tailed unpaired Student's t test. A probability of P<0.05 is considered to be statistically significant for these purposes.

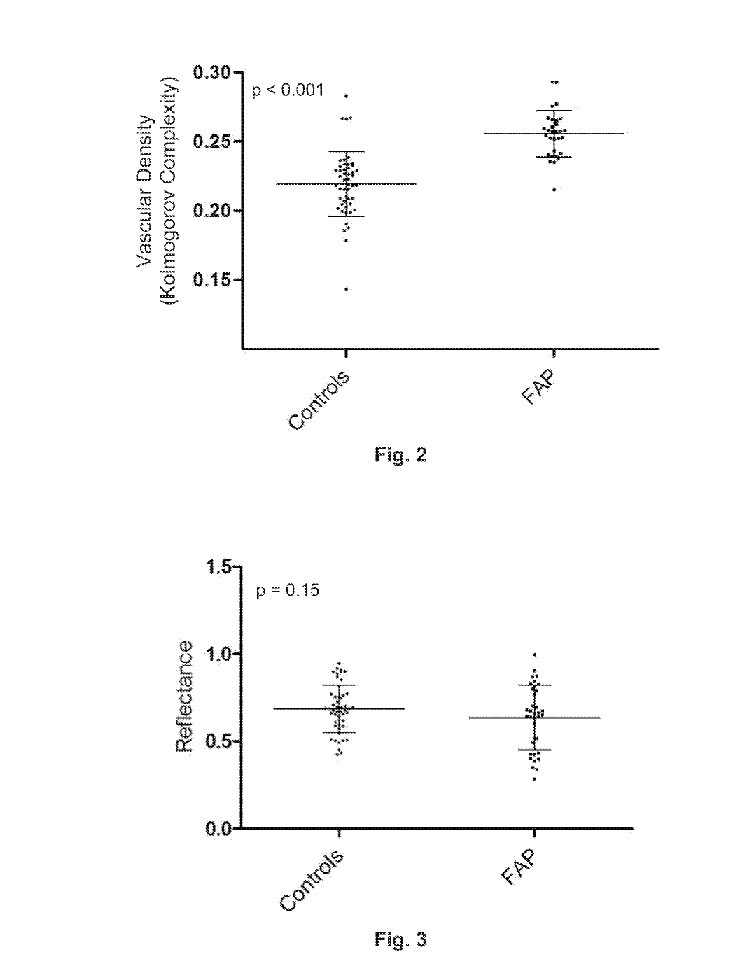

In overall measurements, a difference between vascular density measured in controls versus FAP sufferers which is “diagnostically significant” may be between 5 and 15% (or greater), or between 10 and 15% or greater. For example, as shown in FIG. 2, a vascular density score (p<0.001) calculated by quantifying the Kolmogorov Complexity of traced images (further detailed in the Examples) at about 0.23 on average in controls, whereas the scores in FAP sufferers averaged around 0.27, an approximately 15% increase over the control values.

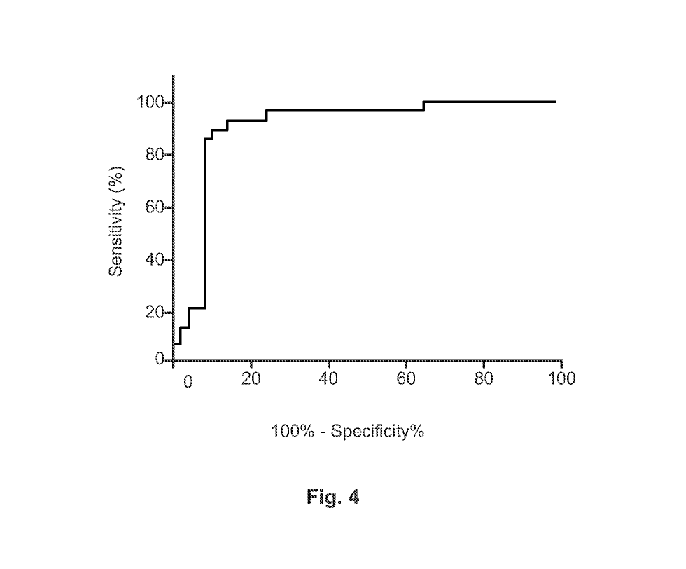

The sensitivity and specificity (collectively, efficacy) of oral mucosal vascular density for FAP determined according to the invention are 90% and 90%, respectively, at a OMVD cut off level of 0.2731 (see, FIG. 4). Sensitivity for FAP is defined as the percentage of affected patients with a positive test (true positives divided by true positives and false negatives expressed as a percentage). Specificity is defined as the percentage of unaffected subjects with a negative test (true negatives divided by true negatives and false positives expressed as a percentage). The predictive value of a positive test is defined as the percentage of subjects with a positive test who had FAP (true positives divided by true positives and false negatives expressed as a percentage). The predictive value of a negative test is defined as the percentage of subjects with a negative test who did not have diagnosed FAP (true positives divided by true positives and false negatives expressed as a percentage). The efficiency of the test is defined as the percentage of all subjects correctly classified (true positives and false positives divided by true negatives and false negatives, expressed as a percentage).

No association between this marker and age or gender was noted. The positive and negative predictive values for oral mucosal vascular density for FAP were 84% and 94%, respectively. Increases in baseline OMVD values for an individual patient, when available, are also considered to be indicative of a risk for developing FAP even if the increase is not to diagnostically significant levels at time of measurement.

If desired to exclude a diagnosis of HNPCC where HCC is suspected, or to exclude a diagnosis of FCC, OMR measurements may also be performed; e.g., through application of the OMR measurement protocol described in DeFelice, et al., Gut, 52:1764-1767, 2003; Parini, et al., Oral Med, 97:335-8 (2004), and elsewhere. Those of ordinary skill in the art will be familiar with or can readily ascertain additional means for performing OMR analysis. Briefly, OMR can be evaluated using an imaging spectrophotometer. The device is calibrated against standard white before each measurement series, and spatially averaged spectra are used to estimate the oral mucosal color in the 400-700 nm wavelength electromagnetic spectral range.

If desired to confirm a diagnosis of FAP made through use of the invention, additional phenotypic markers may be evaluated as elsewhere described hereinabove. Those of ordinary skill in the art will be familiar with or can readily ascertain means for performing such analyses.

It will be appreciated that changes in vascular density may occur both as the disease progresses or as it is ameliorated through therapy. For example, vascular density increases that occurred as a consequence of FAP may be wholly or partially ameliorated or controlled through anti-angiogenic therapy. To this end, an OMVD value obtained in a FAP subject is compared to a later OMVD value from the same subject. An increase, decrease or no difference between the OMVD values is informative regarding the progression of the disease or response to therapy therefor by the subject. Such values may be measured at clinically indicated points over time to monitor the progression of the disease or response to therapy, with such measurements being obtained through practice of the invention.

Kits may be provided for use of the invention which include instructions for performing the OMVD measurements as well as suggestions for confirmatory analyses, such as measurement of OMR to exclude HNPCC as a diagnosis as well as other conventional tests, such as colonoscopy. Optical filters for the image capture device together with instructions for its use would be optionally provided for users who do not have, or may need to replenish, supplies of these items. A copy of or instructions for access to a preloaded version of a numerical analysis program, together with instructions for its use, may also be provided. Further optional additional components may include chin rest covers and forceps or other devices for securing the lip into a suitable position for imaging.

The following examples are intended to illustrate but not limit the invention.

### Example I

### Oral Mucosal Vascular Density Analysis Device

A schematic of an image capture and analysis device useful in the invention is provided in FIG. 1. To provide accurate imaging and reflectance analysis of vessels inside the lower lip of test subjects a device was assembled consisting of a scientific camera (Lumenera Corp., Ottawa, ON, Canada), imaging optics, a computer, and a liquid crystal tunable filter (LCTF), (VariSpec LCTF, Cambridge Research and Instrumentation, Inc., Woburn, Mass.). A program written for MATLAB® controlled the system.

The LCTF has an operational range of 400 to 720 nm in the visible spectrum with a 7 nm bandwidth and utilizes electronically controlled liquid crystal elements to select a transmitted wavelength range while blocking all others. This fine resolution allows for the precise isolation of individual wavelengths, providing a rapid, vibrationless selection of any wavelength in the visible to Near Infrared (NIR) range (470 to 700 nm). The LCTF records reflectance at increments of 5 nm within a range of 500 nm to 700 nm. An 8 bit Lumenera camera mounted to a stereo microscope captured magnified images at a frame rate of 30 fps with a size of 1392×1040 pixels.

The camera was connected to the microscope through an eye-piece optical adaptor, with a ring illuminator (Newport, Irvine, Calif.) positioned in the acquisition side of the microscope assembly. A polarized sheet (Edmund Optics, Barrington, N.J.) was positioned in front of the light source and aligned perpendicularly to the LCTF to eliminate specular reflection. Test subjects were asked to hold their lower lip in a downward position with both hands as the imaging device was aligned to their lower lip. The focal length of the camera remained identical for each subject in order to insure consistency of the imaged area of oral mucosa between patients. Patient movement was largely eliminated through the use of a chin rest, which stabilized the subject in a standardized position. The acquisition and analysis of the data was conducted in MATLAB® on a Pentium 4 laptop (Hewlett Packard® Pavilion).

### Example II

### Oral Mucosal Vascular Density Analysis Technique

78 individuals with a family history of HCC were tested. For confirmation of diagnosis, thirty-three patients with gene positive FAP from 29 unrelated pedigrees, 45 population controls and 5 FAP gene negative patients were recruited at the Gastroenterology department of the Johns Hopkins School of Medicine, exclusion criteria for control patients included anyone with a history of polyps, colon cancer or a first degree relative with colorectal cancer or multiple polyps. Additionally, since no FAP patients were tobacco users, only control patients who were not habitual tobacco users were enrolled.

41 images were acquired from the inside of each patient's lower lip within a timeframe of 25 to 30 seconds and stored as MATLAB® files. Patients were instructed to position their chin inside the chin holder and pull their lower lip downward using both hands over a stabilizer apparatus positioned perpendicular to the chin rest while remaining as still as possible. The imaging field was 1 cm in diameter, though larger imaging samples (2×2 cm) were also considered with no impact to the results. The imaging system was tested and calibrated to determine the most effective gain and exposure time. Exposure time was ultimately set to a relatively short 250 ms throughout the study, which substantially minimized movement artifacts. After each test, patients relaxed their lip for about 1 to 2 minutes before the next set of images were captured.

The images were obtained at multiple wavelengths, starting at an initial wavelength of about 500 nm and increasing in increments of about 5 nm to a final wavelength of about 700 nm.

For reference, OMR was determined in the subjects as well as OMVD. It has been reported that a higher reflectance (above 625 nm) occurs in control subjects than those positive for HNPCC, whereas reflectance below 575 nm was the same for both populations. Thus, the subjects were evaluated by calculating the ratio of reflectance at two wavelengths, 550 nm and 650 nm (R2 and R1, as shown in FIG. 6).

The oral mucosal vascular density was calculated from a 300×600 pixel portion of the main image manually selected by the operator. Each vessel in the image was traced using an automatic tracing algorithm by Sofka and Steward. See Sofka M. and Stewart C. V., “Retinal vessel centerline extraction using multiscale matched filters, confidence and edge measures,” IEEE Transactions on Medical Imaging 25:1531-1546 (2006). A binary map of the traced vessels was generated and inputted into an algorithm quantifying the Kolmogorov Complexity of traced images and calculating an oral vascular density score for each subject. See Kaspar F., and Schuster H. G., “Easily calculable measure for the complexity of spatiotemporal patterns,” Physical Review A 36:842 (2005). The extended template of the multiscale matched filter helps to preserve vessels that are only a pixel wide and usually low contrast.

The oral mucosal reflectance was also calculated from the same 300×600 pixel portion of the main image. The average value of all pixels was calculated, and this value corresponded to the total normal reflectance.

For the investigation of contrast, two regions of interest were selected, both the vessel surface (Vessel_Reflectance) and an avascular background area (Background_Reflectance). The process was repeated for each of the considered wavelengths for 41 subjects, 20 positives and 21 controls. Finally contrast for the complete set of data was calculated as shown in Equation 1, below:

Contrast(└,p)=Background_Reflectance(└,p) [symbol] vesselReflectance(└,p)Background_Reflectance(└,p) (1)

The confidence measure emphasizes the shape of the intensity surface, which helps detect low contrast vessels. The vessel boundary is useful in distinguishing between offset edges near tissue abnormalities and true vessels. Normalized images were input as data into the vessel tracing algorithm.

Finally, the Kolmogorov Complexity of each image was calculated using an algorithm to quantify the degree of OMVD in each binary image (representative data are shown in FIG. 2). The amount of pressure exerted on the lip during measurement was initially thought to cause local ischemia, potentially interfering with the accuracy of vessel tracing. A pressure sensor (Phidgets, Calgary, Alberta, Canada) was embedded on the spacer and connected to a data acquisition card and monitor unit (Fluke 189 RMS multimeter, Everett, Wash., USA) ultimately proving to be unimportant in the final measurement.

OMVD measurements obtained in the patients (0.255±0.017 for FAP patients and 0.219±0.023 for controls) evidenced a statistically significant increase in the OMVD of the former group compared to the latter (FIG. 2). However, OMR measurements obtained in the patients (0.635±0.191 for FAP patients and 0.685±0.135 for controls) showed no statistically significant difference between controls and FAP sufferers (see, FIG. 3).

To evaluate the usefulness of both OMVD and OMR in identifying FAP patients from controls and negatives, the sensitivity (the percentage of affected subjects with a positive test result) and specificity (the percentage of unaffected subjects with a negative test result) were calculated for each test. The sensitivity and specificity were analyzed by receiver operator curve (ROC) characteristics, as reflected in FIG. 4.

Analysis for differences in demographic characteristics between subjects afflicted with familial adenomatous polyposis and controls was done by a two-tailed unpaired Student's t-test. A probability of P<0.05 was considered statistically significant. Receiver operator characteristic (ROC) curves were used to determine the accuracy of oral mucosal vascular density and oral mucosal reflectance levels to discriminate between those affected and unaffected with FAP over a range of cutoff points (as reflected in data provided in FIG. 4).

Sensitivity for FAP was defined as the percentage of affected patients with a positive test (true positives divided by true positives and false negatives expressed as a percentage). Specificity was defined as the percentage of unaffected subjects with a negative test (true negatives divided by true negatives and false positives expressed as a percentage). Predictive value of a positive test was defined as the percentage of subjects with a positive test who had FAP (true positives divided by true positives and false positives expressed as a percentage). Predictive value of a negative test was defined as the percentage of subjects with a negative test who did not have polyposis (true negatives divided by false negatives and true negatives expressed as a percentage). Efficiency of the test was defined as the percentage of all subjects correctly classified (true positives and true negatives divided by true positives and false positives and true negatives and false negatives, expressed as a percentage).

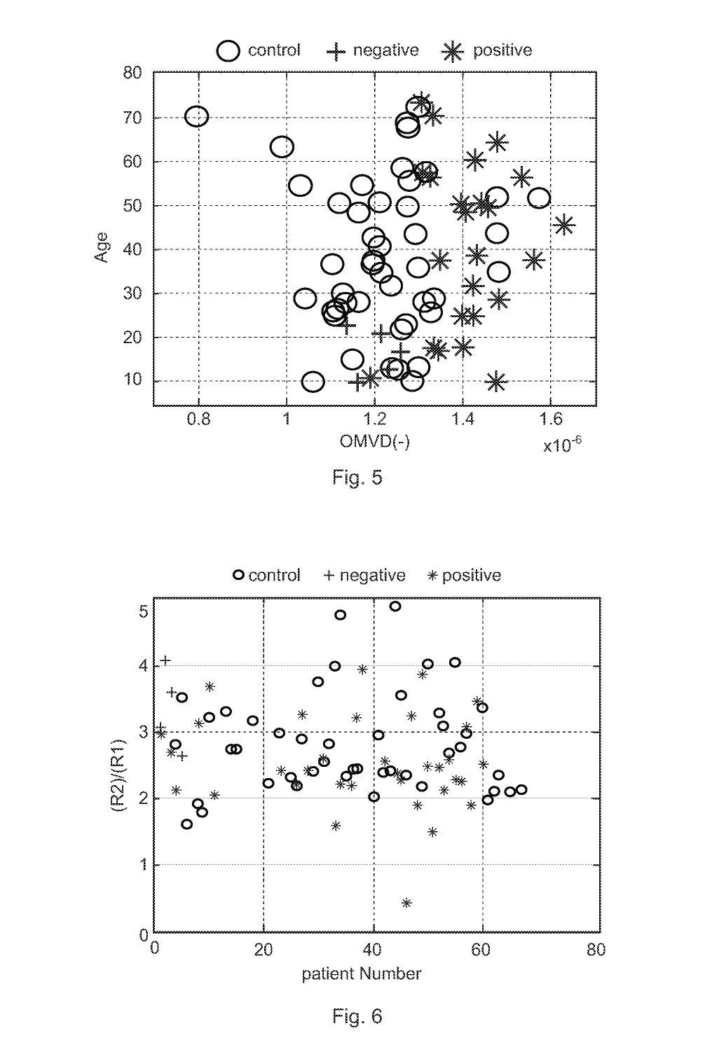

The results obtained show that patients afflicted with familial adenomatous polyposis had statistically significantly increased oral mucosal vascular density compared to controls (p<0.001). The sensitivity and specificity of oral mucosal vascular density for FAP was 90% and 90%, respectively. No association between this marker and age or gender was noted (see, e.g., FIG. 5 as to patient age). The positive and negative predictive values for oral mucosal vascular density for FAP were 84% and 94%, respectively.

Further, with respect to gene positive patients (i.e., those known to suffer from FAP), total diffused oral mucosal reflectance (OMR) and oral mucosal vascular density (OMVD) were calculated from spectral data collected from 33 patients with gene positive FAP, 5 patients who tested negative for FAP, and 45 controls. A statistically significant difference in OMVD (p<0.001) was observed between individuals with FAP and controls. Analysis of OMR showed no significant difference between the two subject groups.

The results herein demonstrate that OMVD is an efficient test for familial adenomatous polyposis which offers a higher level of specificity and sensitivity than available tests or combinations of tests for FAP. It can be performed quickly and non-invasively. In addition, through use of a system such as the one illustrated in FIG. 1, the test is readily susceptible to automation.

All publications, patent applications, patents, and other references are herein incorporated by reference to the same extent as if each publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the invention has been described with reference to the above example, it will be understood that modifications and variations are encompassed within the spirit and scope of the invention. Accordingly, the invention is limited only by the following claims.

1. A method for diagnosing familial adenomatous polyposis (FAP) or an increased risk of FAP in a test subject believed to be at risk for such cancer, the method comprising:
(a) positioning a sampling area of the oral mucosa of the test subject in a system for measuring the oral mucosal vascular density (OMVD);
(b) transilluminating the sampling area of the oral mucosa of the test subject with a light source from the system in a range of wavelengths from 470 nm to 700 nm for a period of time;
(c) obtaining at least one or more images of the illuminated sampling area of the test subject using a camera from the system;
(d) storing the at least one or more images of (c) on an electronic storage device on a computer;
(e) analyzing the at least one or more images of the test subject stored on the electronic storage device to provide an oral mucosal vascular density (OMVD) value from a selected portion of the one or more images; and
(f) comparing the OMVD values of the at least one or more images of the test subject to one or more OMVD values of one or more control subjects which are not afflicted with colorectal cancer; and
(g) diagnosing the test subject has having adenomatous polyposis (FAP) or an increased risk of FAP when there is found a statistically significant increase in OVMD value in the test subject when compared to the one or more control subjects.

2.-6. (canceled)

7. The method of claim 1, further comprising analysis of oral mucosal reflectance (OMR) in the one or more subjects compared to controls.

8. (canceled)

9. The method of claim 7, wherein the difference between the OMR measurements in the one or more subjects does not differ from the OMR measurements in the controls to a statistically significant degree, and confirms the diagnosis of FAP.

10.-14. (canceled)

15. The method of claim 1, wherein the oral mucosa comprises a buccal mucosa.

16. The method of claim 15, wherein the buccal mucosa comprises a lower oral labial vestibular mucosa.

17.-18. (canceled)

19. The method of claim 1, wherein (c) further comprises recording two or more images at multiple wavelengths of light.

20.-27. (canceled)

28. A system for measuring the oral mucosal vascular density (OMVD) value of a subject, the system comprising:
(a) a light source;
(b) magnifying optics;
(c) a tunable filter;
(d) a detector device capable of obtaining one or more images of light reflected from the subject;
(e) an electronic storage device on a computer capable of storing the image of (d);
(f) a numerical analysis program for calculating OMVD from a selected portion of the one or more images wherein the OMVD is calculated by:
(1) tracing each vessel in the mucosa with an automatic tracing algorithm;
(2) generating a binary map of the traced vessels;
(3) inputting the binary map into an algorithm for quantifying a Kolmogorov Complexity of the traced vessels to provide values indicative, of the OMVD of the subject; and
(4) providing OMVD values with a sensitivity and specificity above 80% and up to 90%.

29. The system of claim 28, wherein the detector and the numerical analysis program are in electronic communication for automatic transmission of the image data from the detector to the program.

30. The system of claim 28, wherein the light source is a xenon lamp.

31. The system of claim 28, wherein the light source is coupled to a ring illuminator.

32. The system of claim 28, wherein the tunable filter is a liquid crystal tunable filter.

33. The system of claim 28, wherein the detector device comprises a high-resolution digital optical sensor.

34. (canceled)

35. A method for monitoring the progress of familial adenomatous polyposis (FAP) or treatment therefor in a subject suffering from the disease, the method comprising:
(a) positioning a sampling area of the oral mucosa of the subject in a system for measuring the oral mucosal vascular density (OMVD);
(b) transilluminating the sampling area of the oral mucosa of the subject with a light source from the system in a range of wavelengths from 470 nm to 700 nm for a period of time;
(c) obtaining at least more images of the illuminated sampling area of the subject using a camera from the system;
(d) storing the at least one or more images of (c) on an electronic storage device on a computer;
(e) analyzing the at least one or more images of the subject stored on the electronic storage device to provide an oral mucosal vascular density (OMVD) value from a selected portion of the one or more images; and
(f) comparing the OMVD value of the subjects to a previously measured OMVD value in the same subject, wherein an increase, decrease or no difference between the OMVD values is informative regarding the progression of the disease or response to therapy therefor by the subject.
